(19) United States
(12) Patent Application Publication
(10) Pub. No.: US 2004/0249129 A1
(43) Pub. Date: Dec. 9, 2004

(54) Novel lactic bacteria of the genus lactococcus lactis and use thereof for preserving food products

(76) Inventors: Patrice Daniel, Blain (FR); Sylvie Lorre, Nantes (FR)

Correspondence Address: YOUNG & THOMPSON, 745 SOUTH 23RD STREET, 2ND FLOOR, ARLINGTON, VA, 22202, US

(21) Appl. No.: 10/489,352

(22) PCT Filed: Sep. 18, 2002
(86) PCT No.: PCT/FR02/03180

(30) Foreign Application Priority Data
Sep. 21, 2001 (FR) 01/12187

(51) Int. Cl. G07D 1/00; C07K 1/00; C07K 14/00; C07K 17/00

(52) U.S. Cl. 530/350; 453/35

(57) ABSTRACT

The invention concerns a novel Lactococcus lactis strain called LLO Lactococcus lactis strain and filed at the CNCM (National Micro-organism Culture Collection) under accession number CNCM-127 16. The invention is useful for seafood preservation.

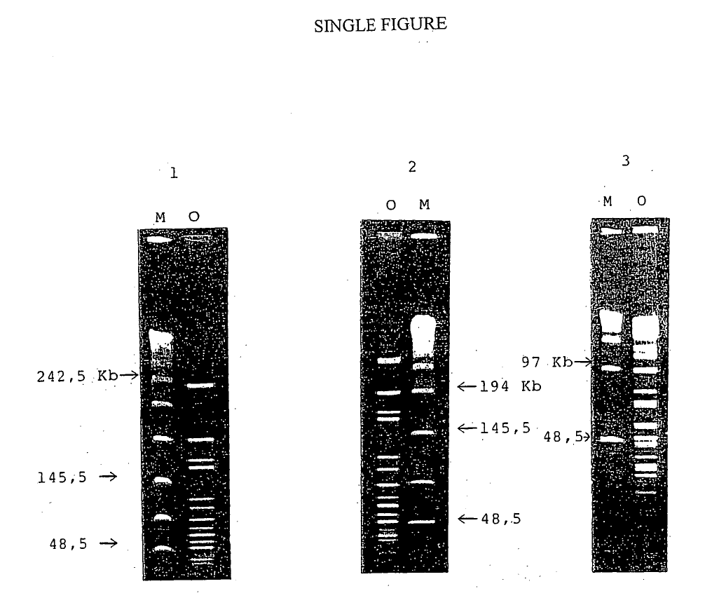

The present invention relates to a new strain of lactic bacteria of the species Lactococcus lactis.

This invention relates moreover to cultures, biomasses, extracts derived from this strain and their use in the preservation of food products.

This invention also relates to compositions incorporating such a strain, to food products treated by means of such a strain and to processes for preservation of such food products.

The present invention is the result of studies carried out in the field of preservation of food products, in particular seafood products packaged under vacuum or in a controlled atmosphere.

Fresh fish is a particularly perishable foodstuff, making its preservation difficult and limiting its distribution. The alteration of refrigerated fresh fish takes place by the development of non-pathogenic microorganisms producing ammonia, amines, hydrogen sulfide... Packaging under vacuum or under a modified atmosphere does not totally inhibit this alteration growth of which a portion is capable of adapting to anaerobic conditions. The result is the development of disagreeable odors which rapidly concentrate, before even the fish taste is changed. “Under vacuum” and modified atmosphere are however the ways of packaging more and more used, because they combine the organoleptic and nutritional qualities of fresh food and a long duration of preservation.

The use of lactic bacteria to preserve refrigerated foodstuff has been the object of various patents or publications.

Thus, Boudreaux et al. in 1989 used live cells of Lactobacillus having the particularity of not fermenting and adapted to produce hydrogen peroxide to inhibit the alteration flora and the pathogenic flora of packaged refrigerated foodstuffs (U.S. Pat. No. 4,874,704). However, hydrogen peroxide was less effective for fish and gives rise to problems as to regulations.

Matrozza et al. (1989) added a mixture of Streptococcus lactis and Pediococcus to a base of non-viable cells to inhibit the psychrotropic flora of refrigerated milk, without obtaining fermentation phenomena or growth of lactic flora (U.S. Pat. No. 4,880,743). This process of inhibition thus relies on the production of active molecules.

Within the scope of a FAR program in 2000, the team of Dr. Hall of the University of Loughborough tested different strains of lactic bacteria isolated from marine products to preserve chopped flesh of whiting. The fermentation amount (strains of Lactococcus or Carnobacterium) had no influence on the organoleptic qualities of the products nor on the inhibition of alteration flora.

Hutkins et al. (1993) introduced into refrigerated products preserved anaerobically, living cells of Pediococcus that produced bacteriocines. The lactic bacteria added did not develop in the foodstuff, nor did they ferment it and did not modify its organoleptic qualities (U.S. Pat. No. 5,186,962). These bacteria-producing bacteriocines proved to be non-viable long-term. Moreover, the bacteriocines are considered as additives of the antibiotic type giving rise to the appearance of resistant germs and thus can lose their effectiveness.

Tests of preservation of smoked salmon with the help of a strain of Lactococcus lactis producing nisine have been carried out by Wessels and Huss in 1996. They noted that the population of Lactococcus lactis decreased in smoked salmon preserved at 10° C., the strain not being psychrotrophic, nor adapted to this type of substrate. The combined action of salt and the quantity of CO₂ permit increasing the bactericidal effects of the nisine against strains of Listeria monocytogenes (Nilsson et al. 1997).

In parallel, the same type of strain has been studied by Chen Mei-Hui et al. Mackerel fillets were cultured by immersion in an acid and salified solution containing a high concentration of Lactococcus lactis cells producing nisine. After drying, the fillets were preserved at 4° C. in bags. The authors were not able to compare the development of alteration flora between seeded and unseeded fillets because the techniques of conventional counting did not permit separating the alteration flora from the added ferment in very great quantity (Chen Mei-Hui et al.: “Processing of low-slated mackerel fillets using biopreservative”. Journal of the Fisheries Society of Taiwan, vol. 24, no. 4, 1997 (1997-12), pages 327-336.

Lactic bacteria isolated from fish, having inhibitory activities as to the alteration flora of fish or strains of Listeria monocytogenes have been the object of different studies (Stoffels et al., 1992; Pilet et al., 1995; Leroi et al., 1996). The studied strains all belong to the genus Carnobacterium. A strain of Leuconostoc isolated from fish also showed aptitude to inhibit strains of Listeria monocytogenes (Jeppensen and Huss, 1993). However, these strains undergo secondary fermentation at 30° C. It is thus impossible to separate the alteration flora from the strain in the course of routine microbiological analyses.

International application WO 00/60947 discloses new strains of bacteria that produce lactic acid. These lactic bacteria are all isolated from milk products and grow at 30° C. None of the bacteria in question alters the foods at temperatures below 7° C. because they do not develop at these temperatures. The bacterial population is divided by 100 after one to two weeks of preservation. The inhibition of pathogenic germs took place only at temperatures above 8° C. It is due to the presence of acids, of carbon dioxide and of bacteriocines or the like. As a result, these lactic bacteria have various drawbacks because they are incapable of inhibiting undesirable germs at temperatures below 8° C. without producing bacteriocine and because they develop at 30° C.

An object of the present invention is to provide a new strain of lactic bacteria adapted to retard the development of bad odors in refrigerated food products, preferably packaged in a controlled atmosphere or under vacuum, and to inhibit the similar germs as well as pathogenic germs to prolong the time of preservation (DLC) of food products, in particular packaged seafood products.

Another object of the invention is to provide a new strain of lactic bacteria incapable of developing at 30° C. so as to permit the counting of the alteration flora of food products during routine microbiological analyses.

To this end, the present invention provides a new strain of lactic bacteria, namely Lactococcus lactis, suitable for use in the field of the preservation of food products, in particular seafood products.

A specimen of the culture of the microorganism, according to the invention, called Lactococcus lactis strain LLO, has been deposited Sep. 11, 2001 under the Treaty of Budapest in the French National Collection of Microorganism Cultures (CNCM), Pasteur Institute—28 rue du Docteur Roux—75724 PARIS CEDEX 15. The specimen received the number CNCM I-2716.

According to this invention, the cultures, biomasses and extracts obtained from the strain mentioned above are also provided. Of course, this invention includes the strains of Lactococcus lactis obtained from mutation, variation, recombination of the Lactococcus lactis strain LLO.

Another object of the invention relates to the use of culture strains, biomasses, extracts of Lactococcus lactis LLO for the preservation of food products as the agent retarding the appearance of bad odors or slowing the development of alteration flora of refrigerated food products and preferably packaged in an atmosphere reduced in oxygen.

Another object of the invention is a food composition for the preservation of the refrigerated condition of food products, in particular seafood products, packaged preferably under an atmosphere reduced in oxygen, characterized in that it comprises at least Lactococcus lactis LLO or a variant or a mutant of the latter.

Another object of the invention is a food product preserved in a refrigerated condition and preferably packaged under an atmosphere reduced in oxygen, characterized in that said product has at its surface a biological barrier constituted at least by Lactococcus lactis LLO or a variant or a mutant of this latter.

Another object of the invention relates to a process for the preservation in a refrigerated condition of food products, in particular seafood products, preferably packaged in an atmosphere reduced in oxygen before consumption, characterized in that it consists in placing in contact the food product to be preserved and Lactococcus lactis preferably in the packaging of said product so as to form a biological barrier at the surface of said product which slows the development of bad odors and the growth of alteration flora of said product.

The invention will be better understood from a reading of the following description of examples of embodiment, with reference to the single attached figure which shows, in the form of three columns, the migrations of different fragments of restriction of the DNA chromosome of the strain LLO.

Lactococcus lactis strain LLO has been isolated from refrigerated fillets of whiting preserved in the presence of a reduced oxygen content.

Lactococcus lactis strain LLO corresponds to the definition of a lactic bacterium. It belongs to the genus Lactococcus species Lactis. Its metabolism in milk or smoked fish does not produce any phenomenon of alteration. It inhibits the alteration flora of whiting, salmon and crustacea.

Morphologically, the cells of Lactococcus lactis strain LLO are ovoidal, immobile. They appear pearwise or in chains.

As to their culture characteristics, these cells are Gram positive, catalase negative, oxydase negative. They do not form spores. They are facultatively anaerobic and have a fermentative metabolism. Fermentation of glucose is accompanied with a slight production of gas. The strain is however homofermentary. It belongs to the genus Lactococcus.

The strain does not hydrolyze arginine, urea or starch, does not produce indole, nor hydrogen sulfide and does not acidify milk. It does not reduce nitrates, it is a hemolytic. It produces acetone.

The strain is psychrotropic and develops between 2 and 25° C. with an optimum growth near 20° C. It is cultivated in the following culture media: MRS pH 6.5, Elliker, M17, Trypticase Soja agar in blood. It does not undergo secondary fermentation in the agar medium of ROGOSA, nor at 30° C. It develops on the flesh of fish or crustacea refrigerated without giving to them odor, nor altered taste.

An antibiogram has been produced with the strain LLO to study its resistance to several antibiotics.

The test is carried out on Mueller Hinton medium containing 1.7% agar and 5% horse blood, the pH is 7.3. The thickness of the agar in the Petri dishes is constant, it is 4 mm. It is a standardized medium according to the O.M.S. standards.

The LLO strain is cultivated in an MRS broth, pH 6.5, for 48 to 36 hours at 15° C. until there is obtained the stationary phase. The culture is diluted to 1/10^{th} in a Mueller Hinton broth. The approximate concentration of the suspension is 10⁶ bacteria/ml.

The agar media are completely covered with 2 ml of the suspension. The excess liquid is withdrawn and the dishes are dried 10 minutes at ambient temperature. The antibiotic discs are deposited with the help of tongs (4 maximum). The dishes are incubated at ambient temperature for 48 hours.

The results are as follows:

| Name | Symbol | Family | Result |
|---|---|---|---|
| Penicillin G | P | Beta-lactamins | Noticeable |
| Ampicillin | AM | | Noticeable |
| Amoxicillin + Acclavulanic | AMC | | Noticeable |
| Cefalexin | CN | | Noticeable |
| Streptomycin (10 | S | Aminosids | Noticeable |
| U.I.) | | | |
| Kanamycin (30 U.I.) | K | | Noticeable |
| Gentamicin (10 U.I.) | GM | | Noticeable |
| Chloramphenicol | C | Phenicols | Noticeable |
| Tetracyclin | TE | Tetracyclins | Noticeable |
| Doxycyclin | DO | | Noticeable |
| Erythromycin | E | Macrolides | Noticeable |
| Spiramycin | SP | | Noticeable |
| Lincomycin | L | | Noticeable |
| Clindamycin | CM | | Noticeable |
| Bacitracin | B | Polypeptides | Noticeable |
| Sulfamid − trimethoprim | SXT | Sulfamides | Resistant |
| Flumequin | UB | Quinolones | Resistant |
| Enrofloxacin | ENR | | Noticeable |
| Furan | FT | Nitrofurans | Resistant |

A genetic identification has been carried out by electrophoresis in a pulsed field after digestion of the genome by a restriction enzyme with occasional sites.

The operative technique is this following:

The bacterial biomass from 10 ml of culture (48 hours 10 in MRS medium, 18° C.) is recovered by centrifugation (10 minutes at 4000 g). The cellular mass is taken up in 10 ml of TE buffer (tris 10 mM, EDTA 1 mM, pH 7.5) then homogenized in a vortex. The DO600 is then taken. Centrifugation is carried out (10 mn at 4000 g) and the mass is taken up in DO600/0.7 ml of T100E buffer (Tris 10 mM, EDTA 100 mM, pH 7.5). The cellular suspension is mixed V/V with LMP agar (low melting point) by melting (2% in TE). It is cooled to 4° C. and plugs of 1 mm thickness are cut out. These are incubated 4 hours at 37° C. in 3 ml of T100E buffer containing 2 mg/ml of lysozyme. The reaction mixture is then removed. It is incubated 16 hours at 37° C. in 3 ml of TESP buffer (tris 10 mM; EDTA 0.5 M; Sarkosyl 10% pH 7.5) containing 3 mg of pronase. After incubation, the plugs are washed 6 times with 10 ml of TE buffer (30 mn each washing) then it is left to digest 4 hours with the restriction enzyme under the following conditions: total volume 120 μl, 30U of enzyme, 1.2 pl of BSA and 1/10 of buffer recommended by the manufacturer OZYME.

After digestion, the plugs are washed in 10 ml of TE buffer (20 mn) then the migration gel is loaded.

The apparatus used is a GeneLine (Beckman).

The enzyme used is Apa I.

Three migration conditions are applied so as to separate under the best circumstances the different restriction fragments obtained. The results are given in the single figure.

The first column corresponds to 10 s of pulse for 16 hours to identify the gross fragments.

The second column corresponds to 7 s of pulse for 16 hours to identify the intermediate fragments.

The third column corresponds to 2 s of pulse for 7 hours then 4 s of pulse for 4 hours to identify the small fragments.

The migration buffer is TBE 0.25 × and the temperature is 12° C.

The results are shown in the single figure.

The inhibitory effects of the LLO strain have been determined in a Petri dish by the double layer technique.

The indicative strains have been cultivated for 24 to 48 hours at 30° C. in broth M17 or MRS for the lactic bacteria and in nutrient broth for the non-lactic, before being seeded into the same medium containing 1% agar. The final concentration of indicative strain is of the order of 10⁷ CFU/ml. This agar maintained melted at 46° C. is poured into a Petri dish on an identical agar containing 1.2% of previously cooled agar. After gelling of the second layer, a drop of mass of the “O” strain cultivated in M17 broth at 20° C. for 3 days and washed in physiological water is deposited on each agar. The dishes are incubated at 30° C.

The scale of sensitivity of the indicative strains relative to the LLO strain is established as follows:

Absence of halo: −

Halo of 1 mm about the deposit: +

Halo of 2 to 3 mm about the deposit: ++ the results obtained are given in the following table:

| Indicative strains | Inhibition by the “O” strain |
|---|---|
| Lc. Lactis ATCC 11 454 | − |
| Staphylococcus spp. | ++ |
| Staphylococcus spp. | ++ |
| Staphylococcus spp. | ++ |
| Bacillus spp/ | − |
| Listeria inocua | + |
| Serratia proteamaculans | ++ |
| Leuconostoc mesenteroides | ++ |
| Pediococcus pentoceus | − |
| Pediococcus acidilactis | − |
| Lactobacillus plantarum | − |
| Lactobacillus farciminis | − |
| Lactobacillus casei | ++ |
| Lactobacillus brevis | − |
| Lactobacillus acidophilus | − |
| Carnobacterium piscicola | + |
| Carnobacterium divergens | − |

The LLO strain is inhibited by the Lactococcus lactis ATCC 11454 strain producing nisine.

Experiment with seeding whiting fillets preserved aerobically have been carried out with the Lactococcus lactis strain LLO. The experiments are described hereafter:

Fillets of whiting have been bought at a fish market. The specimen fillets are arranged in a bag and preserved by refrigeration at 5° C. The test fillets are seeded with the LLO strain by pulverulation of the LLO strain in suspension in physiologic water before being arranged in a second bag and preserved at 5° C. There are carried out on these two types of fillets sensory analyses and microbiological analyses. The sensory analyses are carried out with a sample specimen and a test. The odor and taste after cooking each sample are noted according to a scale of notes as follows:

| | |
|---|---|
| 0 | putrid |
| 1-2 | very bad |
| 3-4 | bad |
| 5-6 | acceptable |
| 7-8 | satisfactory |
| 9-10 | very satisfactory |

The microbiological analyses are carried out particularly by counting the aerobic mesophilic germs. The counting is carried out with removal of 10 g of flesh suspended in 90 ml of physiological sterile water and homogenized for 3 minutes. This suspension is considered as the mother suspension.

Counting the aerobic mesophilic germs that grow at 30° C. for 3 days is carried out by tenfold dilutions of the mother suspension by using the depth counting technique. The culture medium is PCA agar, ordinary agar for the counting in food microbiology.

The LLO strain is counted on MRS agar pH 6.5 by using the depth counting technique, the dishes are incubated 5 days at 15° C.

The results obtained are as follows:

As to the organoleptic tests, the following table gives the results:

| | Days of storage at 5° C. | | |
|---|---|---|---|
| | 0 | 2 | 4 |
| CONTROL | 8/10 | 5/10 | 2/10 |
| TEST | 8/10 | 8/10 | 6/10 |

As to counting, the following table gives the obtained results:

| Days of | | | |
|---|---|---|---|
| storage at | CONTROL | TEST | LLO Strain |
| 5° C. | (cfu/g) | (cfu/g) | (cfu/g) |
| 0 | 13,000 | 13,000 | 7,600,000 |
| 2 | 460,000 | 53,000 | 7,100,000 |
| 4 | 1,800,000 | 340,000 | 8,700,000 |

The experiments carried out permit determining that the food products seeded with Lactococcus lactis strain LLO are always of better organoleptic quality than the controls. There is noted in particular a strong decrease in ammoniacal or hydrogen sulfide odors, permitting an increase in the duration of preservation of treated food products. Regular consumption of seeded food products gives rise to no digestive disorder, nor allergic phenomenon. It is also noted that the development of the usual flora is retarded.

The results obtained thus permit recognizing the possibility of using a composition comprising the Lactococcus lactis strain LLO or a variant or a mutant of the latter, in a food composition for preservation of the refrigerated condition of food products. By refrigeration, there is meant food products preserved at a range of temperature comprised between about 0° C. and 12° C. Conversely, it is noted that this strain does not develop at a temperature of 30° C. This permits counting the flora of the food products in the framework of routine microbiological analyses.

It is also noted that the effect of this strain is increased when it is applied to food products preserved in an atmosphere reduced in oxygen.

Experiments have thus been conducted on fresh Scotch salmon (salmo salar), raised on a farm in the Shetland Isles, eviscerated and iced for 4 days before treatment. This salmon is filleted with the skin. One of the fillets serves as the control, the other the test.

As to the preparation of a control, a fresh control salmon fillet is sliced into eight portions of 100 to 200 g and then packaged in individual bags and placed under vacuum before being stored under refrigeration-between 2 and 4° C.

The test specimen is prepared in the same manner except that it comprises between the step of slicing and the step of packaging a step of seeding by spraying with the LLO strain in suspension in physiological water.

On the basis of these two specimens are carried out on the one hand sensory analyses, on the other hand macrobiological examinations. The sensory analyses are carried out from a control specimen and a test specimen. The color of the flesh is visually monitored. After opening the bags, the odor is noted according to a scale of notes. If necessary, the specimen is cooked in simmering water for several minutes before being again studied.

The scale of notes is established as follows:

| | |
|---|---|
| 0 | putrid |
| 1-2 | very bad |
| 3-4 | bad |
| 5-6 | acceptable |
| 7-8 | satisfactory |
| 9-10 | very satisfactory |

As to microbiological examinations, the pH is measured at different points on the specimens. The value taken is the mean of 3 measurements. Counting is carried out from removal of 10 g of flesh suspended in 90 ml of physiologically sterile water and homogenized for 3 minutes. This suspension is considered as the mother suspension.

The counting of the mesophilic aerobic germs that grow at 30° C. over 3 days is carried out from tenfold dilutions of the mother suspension by using the depth counting technique. The culture medium is PCA agar, ordinary agar for the microbiological counting of foodstuffs.

The LLO strain is counted on MRS agar, pH 6.5, by using the depth counting technique, the dishes are incubated 5 days at 15° C.

The obtained results are as follows:

As to the organoleptic tests, the following table gives the results:

Days of storage after catching

| | 4 | 7 | 11 | 14 |
|---|---|---|---|---|
| CONTROL | 10/10 | 8 and 9/10 | 4/10 | 1/10 |
| TEST | 10/10 | 9 and | 7 and 8/10 | 5/10 |
| | | 10/10 | | |

As to the variations of pH, the following table reproduces the obtained results:

Days of storage after catching

| | 4 | 7 | 11 | 14 |
|---|---|---|---|---|
| CONTROL | 6.4 | 6.3 | 6.5 | 6.2 |
| TEST | 6.2 | 6.3 | 6.2 | 6.3 |

As to counting, the following table gives the obtained results:

| Days of | | | |
|---|---|---|---|
| storage after | CONTROL | TEST | LLO Strain |
| catching | (cfu/g) | (cfu/g) | (cfu/g) |
| 4 | 790 | 790 | 100,000 |
| 7 | 1,200 | 780 | 530,000 |
| 11 | 139,000 | 8,900 | 1,300,000 |
| 14 | 740,000 | 160,000 | 7,400,000 |

The results show clearly that there is no significant difference between the pH of the controls and the treated products. Conversely, the organoleptic qualities of the products as well as the development of common flora give positive results compared to the control specimens.

Other tests have been carried out on farmed shrimp that are beheaded and frozen. These shrimp are thawed in lukewarm water for 5 minutes, before being cooked in salted boiling water at 20 g/l for 1 minute. After rapid cooling under running water, the shrimp are immersed in a salted ice water bath at 20 g/l.

The “control” shrimp are packaged in lots of ten in boxes under modified atmosphere. The “test” shrimp are first seeded with the LLO strain by spraying the LLO strain in suspension in physiological water. They are then packaged in quantities of ten in boxes under modified atmosphere and preserved at 4° C.

Sensory analyses are carried out on the “control” and “test” specimens. The color of the flesh is visually monitored. After opening the packages, the test is noted according to a scale of notes identical to that provided for the example of salmon above.

Counting is carried out by removal of 10 grams of shrimp suspended in 90 ml of physiologically sterile water and homogenized for 3 minutes. This suspension is considered the mother suspension.

The counting of the mesophilic germs, cultivated aerobically at 30° C. for 3 days, is carried out from tenfold dilutions of the mother suspension by using the depth counting technique. The culture medium is PCA agar, the ordinary agar for alimentary microbiological counting.

The LLO strain is counted on MRS agar, pH 6.5, by using the depth counting technique, the dishes are incubated 5 days at 15° C.

As to the organoleptic tests, the following table gives the results:

| | Days of storage at 4° C. | | | | | |
|---|---|---|---|---|---|---|
| | 0 | 5 | 7 | 13 | 21 | 27 |
| CONTROL | 10/10 | 8 and | 8/10 | 7/10 | 7/10 | 6/10 |
| | | 9/10 | | | | |
| TEST | 10/10 | 9 and | 9 and | 9 and | 9/10 | 9/10 |
| | | 10/10 | 10/10 | 10/10 | | |

As to the counting, the following table gives the obtained results:

| Days of | | | |
|---|---|---|---|
| storage at | CONTROL | TEST | LLO Strain |
| 4° C. | (cfu/g) | (cfu/g) | (cfu/g) |
| 0 | 16,000 | 16,000 | 5,600,000 |
| 7 | <300 | <300 | 3,800,000 |
| 13 | 72,000 | <300 | 5,000,000 |
| 21 | 5,800,000 | <300 | 6,400,000 |
| 27 | — | 450 | 7,300,000 |

It is thus possible to envisage an industrial application of this strain in the field of the process of preservation of refrigerated food products. The process would thus consist in placing the food product to be preserved and the Lactococcus lactis strain LLO in contact prior to packaging of the product, in such a way as to form a biological barrier at the surface of the product which would slow the development of bad odors and the growth of alteration flora of the product. Obviously, this contact should be carried out after any treatment of the product, in particular if this latter is to be subjected to a thermal treatment. Placing in contact could be carried out by inoculation of the food product to be preserved with about 10⁵-10⁷ cells remaining viable of Lactococcus lactis strain LLO per gram of product to be preserved. Various methods of inoculation could be envisaged according to the form of preservation of the strain. Thus, the Lactococcus lactis strain LLO could be in lyophilized form. This Lactococcus lactis strain LLO could also be present in the form of a suspension. The seeding of the foodstuff could also be carried out by spraying a powder incorporating the Lactococcus lactis strain LLO, or by spraying a cellular suspension incorporating the mentioned strain, or by immersion of the foodstuff in a bath containing the strain. The strain could be used alone or mixed with a support.

This strain thus has the properties:

of developing on seafood products, such as fish, crustacean or the like, at refrigeration temperatures,

not altering the foodstuff,

permitting, under certain circumstances, slowing the development of the common flora of alteration of the foodstuff and inhibiting certain pathogens,

retarding the appearance of undesirable odors, such as ammoniacal or hydrogen sulfide odors.

Its efficacy is increased when the foodstuff is packaged under vacuum, under a modified atmosphere or any other packaging reducing the presence of oxygen.

It does not develop at 30° C. and can be counted in a usual microbiological analysis of foodstuff.

1. New strain of Lactococcus lactis called Lactococcus lactis strain LLO and deposited at the CNCM under the accession number CNCM I-2716.

2. Any strain of Lactococcus lactis obtained by mutation, variation, recombination of the strain according to claim 1.

3. New strain of Lactococcus lactis LLO according to claim 1,
characterized in that it has a growth temperature comprised between 2° C. and 25° C. with an optimum growth temperature of about 20° C.

4. Cultures, biomasses and extracts obtained from strains according to claim 1.

5. Composition, characterized in that it comprises at least the Lactococcus lactis strain LLO or a variant or a mutant of the latter according to claim 1.

6. Composition according to claim 5, characterized in that the Lactococcus lactis strain LLO is in lyophilized form.

7. Composition according to claim 5,
characterized in that the Lactococcus lactis strain LLO is in the form of a suspension.

8. Food composition for the preservation in refrigerated condition of food products, in particular of seafood products, packaged preferably under an atmosphere reduced in oxygen,
characterized in that it comprises the Lactococcus lactis strain LLO according to claim 1 or a variant or a mutant of this latter, particularly as an active agent retarding the appearance of bad odors and/or the development of bacteria that are pathogenic or not.

9. Food product preserved in the refrigerated condition and packaged preferably under an atmosphere reduced in oxygen,
characterized in that said product has at its surface a biological barrier constituted at least of Lactococcus lactis LLO or a variant or a mutant of this latter according to claim 1.

10. The use of strains, cultures, biomasses, extracts of Lactococcus lactis LLO according to claim 1 as an agent retarding the appearance of bad odors in food products preserved in refrigerated condition and packaged preferably under an atmosphere reduced in oxygen.

11. The use of strains, cultures, biomasses, extracts of Lactococcus lactis LLO according to claim 1 as an agent slowing the development of alteration flora of refrigerated foodstuffs packaged preferably under an atmosphere reduced in oxygen.

12. Process for the preservation in refrigerated condition of food products, in particular seafood products, packaged in an atmosphere reduced in oxygen before consumption,
characterized in that it consists in placing in contact the food product to be preserved and Lactococcus lactis LLO according to claim 1 prior to packaging said product so as to form at least one biological barrier at the surface of said product which slows the development of bad odors and the growth of alteration flora of said product.

13. Process according to claim 12,
characterized in that the food product to be preserved and Lactococcus lactis LLO are placed in contact by inoculation of the product to be preserved with about 10⁵-10⁷ cells remaining viable of Lactococcus lactis LLO per gram of product to be preserved.

14. New strain of Lactococcus lactis LLO according to claim 2,
characterized in that it has a growth temperature comprised between 2° C. and 25° C. with an optimum growth temperature of about 20° C.

15. Cultures, biomasses and extracts obtained from strains according to claim 2.

16. Process for the preservation in refrigerated condition of food products, in particular seafood products, packaged in an atmosphere reduced in oxygen before consumption,
characterized in that it consists in placing in contact the food product to be preserved and Lactococcus lactis LLO according to claim 2 prior to packaging said product so as to form at least one biological barrier at the surface of said product which slows the development of bad odors and the growth of alteration flora of said product.

17. Process for the preservation in refrigerated condition of food products, in particular seafood products, packaged in an atmosphere reduced in oxygen before consumption,
characterized in that it consists in placing in contact the food product to be preserved and Lactococcus lactis LLO according to claim 3 prior to packaging said product so as to form at least one biological barrier at the surface of said product which slows the development of bad odors and the growth of alteration flora of said product.
